(19) United States
(12) Patent Application Publication
(10) Pub. No.: US 2005/0164952 A1
(43) Pub. Date: Jul. 28, 2005

(54) Delivery system for growth hormone releasing peptides

(75) Inventor: John H. Owoc, Davie, FL (US)

Correspondence Address: LERNER AND GREENBERG, PA, P O BOX 2480, HOLLYWOOD, FL, 33022-2480, US

(73) Assignee: Vital Pharmaceuticals, Inc.

(21) Appl. No.: 11/041,575

(22) Filed: Jan. 24, 2005

Related U.S. Application Data
(60) Provisional application No. 60/539,002, filed on Jan. 23, 2004.

(51) Int. Cl. A61K 38/08

(52) U.S. Cl. 514/17

(57) ABSTRACT

A system for delivering a growth hormone releasing peptide including a peptide, or a pharmaceutically acceptable salt form thereof, alternatively conjugated to a polymer via a biodegradable bond and dissolved in a liquid medium. Because extra-cellular fluids in vivo provide the appropriate environment to efficiently degrade peptides, the growth hormone releasing peptide will maintain chemical integrity and remain substantially coupled to the polymer while circulating through the body. The therapeutic agent is, therefore, protected from degradation and clearance.

### CROSS REFERENCE TO RELATED APPLICATION

This application claims the benefit under 35 U.S.C. § 119 (e), of copending U.S. Provisional Application No. 60/539,002, filed Jan. 23, 2004.

### BACKGROUND OF THE INVENTION

Growth hormone (GH) is an anabolic hormone capable of promoting linear growth, weight gain, and whole body nitrogen retention. GH is thought to be released primarily from the somatotroph cells of the anterior pituitary under the coordinate regulation of two hypothalamic hormones: (i) an excitatory 44-amino acid peptide referred to as “growth hormone releasing hormone” (GHRH) and (ii) a tetradecapeptide hypothalamic hormone known as “somatostatin” (SS). Both GHRH stimulation and SS inhibition of the release of GH occur by the specific engagement of receptors on the cell membrane of the somatotroph.

GH secretion is known to be stimulated by GHRH, and inhibited by SS, in all mammalian species, including humans. It is believed that episodic secretion of GH is caused by the rhythmic, alternating release of GHRH and SS, both of which are regulated through the so-called pituitary-hypothalamus axis. Secreted GH, both directly and indirectly through an insulin-like growth factor (IGF-1), appears to maintain this regulation by, in turn, stimulating SS and inhibiting further GHRH release. Other neurotransmitters are also believed to modulate GH, usually by stimulating or inhibiting SS. Additionally, other factors including exercise, sleep, glucocorticoids, thyroid hormones, sex steroids, free fatty acids, amino acids, and glucose levels further modulate GH release.

In the early 1980s, it was discovered that GH release is also stimulated by a group of short peptides referred to as the “Growth Hormone Releasing Peptides” (GHRP). It is believed that these peptides function by selectively binding to a distinct somatotroph cell membrane receptor, the “Growth Hormone Secretagogue Receptor” (GHSR). Typically, GHRPs are partial peptides consisting of natural and unnatural amino acids of varying chain lengths. One such peptide is a hexapeptide known as GHRP-2, for example, hexarelin, described in International PCT Patent Publication No. WO 93/04081 (corresponding to U.S. Pat. No. 5,663,146, U.S. Pat. No. 5,776,901, and U.S. Pat. No. 4,880,777).

Because the GHRPs have been the most effective in promoting elevated growth hormone levels, they have had a number of clinical applications. For example, GHRPs have been administered to stimulate growth hormone production and/or release to stimulate growth, enhance milk production, enhance body weight, increase rate of protein synthesis, reduce rate of carbohydrate utilization, and increase mobilization of pre-fatty acids.

Notwithstanding these beneficial effects, the protein nature of growth hormone peptides has made anything but parenteral administration non-viable because proteases and endonucleases present throughout the body digest such compounds, severely decreasing their biological activity. Other problems involve the rapid clearance of the compounds or fragments thereof, particularly if the parent agent has a low molecular weight. Hence, in order to be effective, these therapeutic agents must be administered frequently, and parenterally, rather than orally.

In efforts to overcome these problems, researchers have attempted to modify chemically such therapeutic agents in order to manipulate their pharmacologic properties, and perhaps enable them to survive longer in vivo before being degraded and removed from the blood stream. However, such modifications have inherent limitations. For example, they frequently interfere with the bioavailability of the therapeutic agent, or otherwise deleteriously affect the biological activity of the agent.

### SUMMARY OF THE INVENTION

It is therefore desirable to find improvements in the design and delivery of these important compounds. For example, an improved GHRP would maintain its growth hormone releasing effects, while having at least one other desirable biological activity or chemical property such as better bioavailability, absorption, metabolism, pharmacokinetics, excretions, duration, rate of clearance, enhanced stability, etc. It would also be desirable to have a delivery system to use in connection with GHRPs, which could further promote these additional qualities. Such discoveries of optimum physical-chemical properties and physiological-biological actions could make various diagnostic and therapeutic uses in humans realistic upon oral delivery.

Accordingly, there is provided a system for in vivo delivery of therapeutic agents that do not possess the shortcomings of other drug delivery approaches, and offers the advantages of enhanced bioavailability of the therapeutic agent, and protection from degradation and rapid clearance, to name only a few.

It is therefore an object of the present invention to provide novel compounds with growth hormone releasing properties, wherein the growth hormone releasing compound is a peptide, or an ester thereof, or pharmaceutically acceptable salts thereof, wherein the compounds may be delivered in a suitable delivery system in vivo.

It is a further object of the present invention to provide novel compounds with growth hormone releasing properties which have one or more of improved properties, including improved bioavailability, increased stability, a lower rate of clearance, or other beneficial chemical or biological properties.

It is a further object of the present invention to provide a system for in vivo delivery of a therapeutic agent wherein the therapeutic agent is conjugated to the carrier to improve one or more of bioavailability, stability, or rate of clearance, and is suitably formulated.

It is another object of the invention to provide a method of modulating growth hormone release in a mammal, preferably a human, which includes administering a therapeutically effective amount of the compounds and compositions of the present invention in a suitable delivery system. These, and other objects, will become apparent during the following detailed description.

### DETAILED DESCRIPTION OF THE INVENTION

Peptides are often unable to adequately enter the bloodstream in efficacious amounts due to their poor solubility characteristics, electronic properties and the like.

Moreover, because peptides are modified through metabolism, they may be broken down into smaller level peptides, or amino acids, and subjected to first pass liver clearance. These, as well as other obstacles, impede the ability of peptides to cross the blood brain barrier intact, in turn, limiting their ability to induce the anterior portion of the pituitary gland into increasing endogenous production of bioactive growth hormone. Therefore, the longer a peptide can survive intact in the body, the more pronounced effect the compound will have on its ultimate target.

It has been unexpectedly discovered that the pharmacokinetic and pharmacodynamic limitations of growth hormone releasing peptides may be overcome by modifying the structure of the peptide itself, and the form and manner in which it is introduced to the subject. In this connection, peptides, their esters, and salts thereof, alternatively conjugated with certain long chain polymers, and encased in a lipid or other excipient-based delivery systems, are suitable for oral delivery, and are expected to induce improved blood levels over an extended period of time, or possess other beneficial properties.

Thus, in a first embodiment, the present invention provides a growth hormone releasing peptide or a pharmaceutically acceptable salt form thereof. These peptides may exist as free acids, zwitterions, or organic and inorganic salt forms. In certain embodiments, the growth hormone releasing peptide is a hexapeptide. In certain embodiments, the hexapeptide is D-Ala-D^{β}Nal-Ala-Trp-D-Phe-Lys. In certain embodiments, the peptide exists as an acetate or other acid addition salt.

In another embodiment, the present invention provides a growth hormone releasing peptidic ester or a pharmaceutically acceptable salt form thereof. In certain embodiments, the growth hormone releasing peptidic ester is a C₁-C₁₀ alkyl, aryl, alkylaryl or acetate ester. In certain embodiments, the ester is a short chain or branched C₁-C₁₀ alkyl ester, such as methyl, ethyl, propyl, iso-propyl, butyl, sec-butyl, t-butyl, etc. In certain embodiments, the growth hormone releasing peptidic ester is a hexapeptide. In certain embodiments, the hexapeptidic ester is D-Ala-D^{β}Nal-Ala-Trp-D-Phe-Lys-OR, wherein R is a group that cleaves to yield the free hydroxyl after being administered to a subject, for example, in vivo. In certain embodiments, the peptidic ester is an acetate or other acid addition salt.

In another embodiment, the present invention provides a pharmaceutically acceptable composition, referred to herein as a system for the delivery of a growth hormone releasing peptide (i.e., “delivery system”), containing a growth hormone releasing compound in a pharmaceutically acceptable formulation. In certain embodiments, the delivery system includes a growth hormone releasing peptide, an ester thereof, or a pharmaceutically acceptable salt form thereof, in a formulation containing at least one pharmaceutical excipient. In certain embodiments, the delivery system consists of one or more excipients selected from the group consisting of water, lipids, liposomes, polymers, polysaccharides, organic solvents, organic acids, mineral acids, surfactants, sweeteners, flavorants, stabilizers, viscosity agents, preservatives, emulsifiers, anti-oxidants, solubilizers, fats and gums, to name a few. In certain embodiments, the excipients are selected from the group consisting of potassium sorbate, sodium benzoate, soy lecithin, safflower oil, PEG-40 stearate, ethyl alcohol, polysorbate 80, citric acid, chitosan, and phosphoric acid. In certain embodiments, the delivery system includes the salt of a growth hormone releasing peptidic ester. In certain embodiments, the delivery system includes the salt of a growth hormone releasing peptide. In certain embodiments, the salt is an acetate or other acid addition salt. In certain embodiments, the growth releasing peptide is a hexapeptide. In certain embodiments, the growth releasing peptide is the ester of a hexapeptide. In certain embodiments, the hexapeptide ester is a salt of D-Ala-D^{β}Nal-Ala-Trp-D-Phe-Lys-OR, wherein R is a group that cleaves to form a free hydroxyl after being administered to a subject, for example, in vivo. In certain embodiments, R is a C₁-C₁₀ alkyl, aryl, alkylaryl or acetate ester.

In certain embodiments, the delivery system includes the following: (a) a growth hormone releasing peptide, ester thereof, or a pharmaceutically acceptable salt form thereof and (b) a lipid. In certain embodiments, the delivery system includes the following: (a) a polymer; (b) a growth hormone releasing peptide or ester thereof or a pharmaceutically acceptable salt form thereof, optionally conjugated to the polymer; and (c) a lipid. In certain embodiments, the delivery of the growth hormone releasing peptide occurs in-vivo. In certain embodiments, the growth releasing peptide or ester thereof, once administered to a mammalian subject, cleaves to form a free hydroxyl. In certain embodiments, the ester is an ester of a hexapeptide. In certain embodiments, the hexapeptide is D-Ala-D^{β}Nal-Ala-Trp-D-Phe-Lys. In certain embodiment, a second growth releasing peptide ester is conjugated to the polymer. In certain embodiment, the second peptide ester is D-Ala-D^{β}Nal-Ala-Trp-D-Phe-Lys. In certain embodiments, the polymer is an ethylene polymer. In certain embodiments, the lipid is a phospholipid. In certain embodiments, the delivery system further includes water and a surfactant.

In another embodiment, the present invention provides a pharmaceutical composition including the delivery system described herein.

In another embodiment, the present invention provides a method of forming a system for the delivery of a growth hormone releasing peptide or ester thereof, wherein the method includes: conjugating a growth hormone releasing peptide to a polymer; and dispersing the conjugated peptide ester in a lipid to form a delivery system. In certain embodiments, the method further includes contacting the delivery system with an aqueous medium in vivo. In certain embodiments, the growth hormone releasing peptide is the free acid, zwitterion, or an ester of D-Ala-D^{β}Nal-Ala-Trp-D-Phe-Lys. The delivery system may further include a surfactant and water.

In another embodiment, the present invention provides a method of promoting the release and elevation of growth hormone levels in humans that includes administering to a human a therapeutically effective amount of the compounds and/or the delivery system described herein.

In another embodiment, the present invention provides a method for forming a system for delivering a growth hormone releasing peptide, which comprises (1) providing a growth releasing peptide or a pharmaceutically acceptable salt form thereof; and (2) dispersing the peptide in one or more pharmaceutically acceptable excipients under suitable conditions to form a delivery system.

In another embodiment, the present invention provides a method of releasing and elevating growth hormone levels, which comprises administering a therapeutically effective amount of the compounds and/or delivery system described herein. In certain embodiments, the release of growth hormone improves muscle strength, improves mobility, stimulates growth hormone release in elderly humans, prevents catabolic side effects of glucocorticoids, prevents osteoporosis, stimulates the immune system, and accelerates wound healing.

The terms used in the claims and specification are defined as set forth below unless otherwise specified.

As used herein, the term “growth hormone releasing hormone” or “GHRH” refers to the endogenous hypothalamic growth hormone secretagogue, having the capability of binding to the pituitary somatotroph and inducing a rapid dose-dependent release of growth hormone.

As used herein, the term “somatostatin” refers to the inhibitory hypothalamic tetradecapeptide capable of antagonizing in a dose-dependent manner the GH-releasing effect of GHRH.

As used herein, “IGF-1” refers to insulin-like growth factor, preferably human.

As used herein, “amino acid” is intended to have its art-recognized meaning, i.e., a carboxylic acid of general formula HOC(═O)CH(side chain)(NH₂). Side chains of amino acids are well known in the art and include naturally occurring and non-naturally occurring moieties. Non-naturally occurring (i.e., unnatural) amino acid side chains are moieties that are used in place of naturally occurring amino acid side chains in, for example, amino acid analogs.

The residues of amino acids described herein are in agreement with standard nomenclature, for example, those set forth as follows:
Gly Glycine
Tyr L-Tyrosine
Ile L-Isoleucine
Glu L-Glutamic Acid
Thr L-Threonine
Phe L-Phenylalanine
Ala L-Alanine
Lys L-Lysine
Asp L-Aspartic Acid
Cys L-Cysteine
Arg L-Arginine
Ava Aminovaleric acid
Aib Aminoisobutyric acid
Gln L-Glutamine
Pro L-Proline
Leu L-Leucine
Met L-Methionine
Ser L-Serine
Asn L-Asparagine
His L-Histidine
Trp L-Tryptophan
Val L-Valine
DOPA 3,4-Dihydroxyphenylalanine
Met(O) Methionine Sulfoxide
Abu α-Aminobutyric Acid
iLys N^{ε}-Isopropyl-L-Lysine
4-Abu 4-Aminobutyric Acid
Orn L-Ornithine
D^{α}Nal α-Naphthyl-D-Alanine
D^{β}Nal β-Naphthyl-D-Alanine
Sar Sarcosine
LArg homoArgininine

Although the amino acid residues described herein are preferred to be in the “L” isomeric form, all of the three letter-abbreviations of the amino acids preceded by a “D” indicate the dextro-isomer of the aminoacidic residue, and glycine is considered included in the term naturally occurring L-amino acids.

As used herein, “peptide” is intended to have its art recognized meaning, i.e., two or more amino acids linked through amide bonds, for example, repeating units of formula —C(═O)CH(side chain)NH— that, in the simplest form, terminate in either an amine or a carboxylic acid. As one of ordinary skill in the art will recognize, numerous modifications of the peptidic backbone are possible without changing the overall nature of the molecule, including modification of the terminal groups such as those described herein.

As used herein, the term “growth hormone releasing peptide” or “GHRP” refers to peptides that cause release of endogenous grown hormone in a dose-dependent manner. Accordingly, “growth hormone releasing peptide” includes the free acid of any given peptide, or any derivatives thereof, such as esters.

Accordingly, as used herein, “growth hormone releasing peptide ester” (or “peptidic ester”) is intended to mean a growth hormone releasing peptide in which one of the acid groups has been chemically converted to an ester.

As used herein, the term “polymer” encompasses both homopolymers and copolymers, whether water soluble and water insoluble.

As used herein, “lipid” encompasses a fat, oil, wax, sterol, glycerol ether, triglyceride, or combination thereof. As used herein, “phospholipids” encompasses phosphatidylcholine, phosphatidylglycerols, ethanolamines, sphingomyelines, phosphatidylserine, dipalmitoylphosphatidylglycerol, dipalmitoylphosphatidylcholine, phosphatidylethanolamine, alpha-tocopherol polyethylene glycol succinate, amphilic surface-active agents, a combination thereof, or any other phospholipids known in the art.

As used herein, “liposome” encompasses substances derived from phospholipids or other lipid substances formed by mono- or multi-lamellar hydrated liquid crystals that are dispersed in an aqueous medium. Liposome encompasses mono or multi-lamellar vesicles derived from liquid crystalline substances such as phospholipids or other lipids that are dispersed in an aqueous medium. As will be appreciated by one of ordinary skill in the art, depending upon the composition, dispersions, micelles, emulsions, and the like may result. For example, such mixtures may result upon contacting an amphiphilic substance like a phospholipid with water. Accordingly, all such variations are contemplated by the term “liposome.”

As used herein, “therapeutically effective amount” refers to an amount of compound effective to prevent or treat the symptoms of a particular disorder.

As used herein, the term “bioavailability” as used herein refers to the ability of a therapeutic agent to enter the bloodstream and reach its biological target following oral administration.

As used herein, the term “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio.

As used herein, “pharmaceutically acceptable salts” refer to derivatives of the disclosed compounds wherein the parent compound is modified by making acid or base salts thereof.

The present invention extends to in vivo delivery of a therapeutic agent. Although the compositions described herein may conceivably improve any therapeutic substance with poor aqueous solubility characteristics, the described compositions preferably contain a peptide, a salt form thereof, preferably present in the composition in a therapeutically effective amount to modulate the production of growth hormone. Typically, this involves affecting the activity of target receptors associated with the production thereof, wherein the administration of active substances inhibits, induces, or enhances the activity of the receptor when it is contacted with the active substance.

Preferably, the active substances will be those that have improved properties as compared to growth hormone releasing proteins previously known or in the absence of a delivery system. In this regard, certain forms of peptides, for example, esters, are expected to enhance numerous desirable qualities including without limitation, bioavailability, absorption, metabolism, pharmacokinetics, excretions, duration, rate of clearance and the like. Certain forms of peptides, for example, salts, are also expected to enhance numerous desirable qualities, including without limitation, solubility, stability, crystallinity, physical form, polymorphism, as well as imparting other manufacturing benefits such as ease of handling and the like. When the aforementioned peptide forms are presented in an appropriate delivery system containing the excipients described herein, the system presents additional benefits, including without limitation, ease of delivery, mode of administration, dosage size, stability of formulation, and the like. Certain aspects of the instant invention may also have synergistic effects. Thus, the present invention relates generally to the parent acids or esters of the described peptides, including the salts thereof, peptide esters conjugated to polymers, compositions containing the same, and methods of delivering the same.

The active peptide may be, for example, twenty amino acids or less. Preferably, the peptide will be less than ten amino acids, but more than three amino acids. More preferably, the peptide will have five or six amino acids. Such peptides include without limitation:
D-Ala-D^{β}Nal-Ala-Trp-D-Phe-Lys, 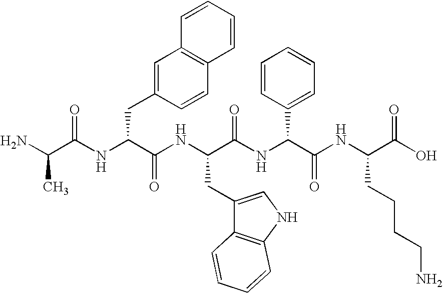
   and pharmaceutically acceptable salt forms thereof.

The growth hormone peptide esters may be any peptide containing an ester functionality, for example, at the terminal acid position of the peptides described and shown above. Preferably, the peptidic ester is an ester of a five or six amino acid peptide. Such peptides include without limitation:
D-Ala-D^{β}Nal-Ala-Trp-D-Phe-Lys-OR,
D-Ala-D^{β}Nal-Trp-D-Phe-Lys-OR
   and pharmaceutically acceptable salt forms thereof, wherein OR indicates that the amino acid to which it is attached is in the ester form instead of acid form, such that —OR is attached directly to the carbonyl carbon of the parent acid. As will be appreciated by those skilled in the art, R may be any group that, once administered to a mammalian subject, the OR groups hydrolyzes or cleaves to form a free hydroxyl (OH). Examples include, but are not limited to, C₁-C₁₀ alkyl, carboxylic, aryl, and alkylaryl esters such as methyl, ethyl, propyl, isopropyl, butyl, isobutyl, sec-butyl, tert-butyl, cyclopropyl, cyclobutyl, cyclopentyl, cyclohexyl, phenyl, benzyl, and phenethyl esters, and the like. Preferably, R is selected from methyl, ethyl, propyl, isopropyl, t-butyl, cyclopropyl, cyclopentyl, cyclophenyl, benzyl, phenethyl. More preferably, R is methyl, ethyl or OOCCH₃ (the methyl, ethyl or acetate ester).

The peptides of the present invention are commercially available, or may be prepared by methods known to those of ordinary skill in the art. For example, one method of chemically producing peptide backbones involves methodologies well known to those skilled in the art, including those described in U.S. Pat. Nos. 5,663,146, 4,105,603; 3,972,859; 3,842,067; and 3,862,925.

Compounds of the present invention may be conveniently prepared using solid phase peptide synthesis using standard protection/deprotection strategies as described by Merrifield, J. Am. Chem. Soc., 85: 2149 (1964); and Houghten, Proc. Natl. Acal. Sci USA 82: 5132 (1985). By way of illustration, solid phase synthesis may begin at the carboxy-terminus of the putative peptide by coupling a protected amino acid to a suitable resin, for example, chloromethylated polystyrene resin, benzhydryl amine (BSA), or para-methyl-benzylhydrylamine (p-Me-BSA). After removal of the α-amino protecting group with, for example, trifluoroacetic acid (TFA) in methylene chloride and subsequent neutralization, the next α-amino (and if necessary, side-chain) protected amino acids are added under standard condensation coupling conditions. The terminal groups are then suitably deprotected and are ready for further coupling. The remaining suitably protected amino acids are then coupled sequentially in the desired order to obtain an intermediate compound connected to the resin. Alternatively, any combination of amino acids may be coupled to one another to form a peptide intermediate chain before addition of the peptide to the growing solid phase polypeptide chain.

The coupling of two amino acids, or an amino acid and a peptide, or a peptide and a peptide can be carried out according to the usual condensation conditions such as the azide method, mixed acid anhydride method, DCC(N,N′-dicyclohexylcarbodiimide) or DIPC(N,N′-diisopropylcarbodiimide)methods, active ester method (p-nitrophenyl ester method, BOP [benzotriazole-1-yl-oxy-tris (dimethylamino) phosphonium hexafluorophosphate] method, N-hydroxysuccinic acid imido ester method, etc., and Woodward regent K method.

Common to chemical syntheses of peptides is the protection of any reactive side-chain groups of the amino acids with suitable protecting groups. Ultimately these protecting groups are removed after the desired polypeptide chain has been sequentially assembled. Also common is the protection of the α-amino group on an amino acid or a fragment while that entity reacts at the carboxyl group followed by the selective removal of the α-amino-protecting group to allow subsequent reaction to take place at that location. Accordingly, it is common in polypeptide synthesis that an intermediate compound is produced which contains each of the amino acid residues located in the desired sequence in the peptide chain with various of these residues having side-chain protecting groups attached. These protecting groups are then commonly removed substantially at the same time to produce the desired resultant product following removal from the resin.

Suitable protective groups for protecting amino side chain groups will be readily understood by the skilled artisan, and are exemplified by benzyloxycarbonyl (CBZ), isonicotinyloxycarbonyl(iNOC), O-chlorobenzyloxycarbonyl (2-Cl-CBZ), p-nitrobenzyloxycarbonyl [Z(NO₂)], p-methoxybenzyloxycarbonyl [Z(OMe)], t-butoxycarbonyl, (BOC), t-amyloxycarbonyl (AOC), isoborrnyloxycarbonyl, adamatyloxycarbonyl, 2-(4-biphenyl)-2-propyl-oxycarbonyl (BPOC), 9-fluorenylmethoxycarbonyl (FMOC), methylsulfonyiethoxycarbonyl (Msc), trifluoroacetyl, phthalyl, formyl, 2-nitrophenylsulphenyl (NPS), diphenylphosphinothioyl (Ppt), dimethylophosphinothioyl (Mpt) and the like. Protective groups for the carboxy functional group are exemplified by; benzyl ester (OBzl), cyclohexyl ester (Chx), 4-nitrobenzyl ester (ONb), t-butyl ester (Ot-Bu), 4-pyridylmethyl ester (OPic), and the like. It is often desirable that specific amino acids possessing a functional group other than amino and carboxyl groups are protected by a suitable protective group. Other preferred protecting groups according to the invention may be found in Greene, T. W. and Wuts, P. G. M., Protective Groups in Organic Synthesis 2d. Ed., Wiley & Sons, 1991, the disclosure of which is hereby incorporated herein by reference in its entirety.

After the desired amino acid sequence has been completed, the intermediate peptide is removed from the resin support by treatment with a reagent, such as, for example, liquid HF, which may, if desired, not only cleave the peptide from the resin, but also cleave all the remaining side-chain protecting groups. Following cleavage, the peptide residue may be washed with ether, and extracted from the resin by washing with solvent, for example, aqueous acetonitrile and acetic acid. Preferably, in order to avoid alkylation of residues in the polypeptide (for example, alkylation of methionine, cysteine, and tyrosine residues), a scavenger mixture may be used, such as thio-cresol and cresol.

The esters of the present invention may be afforded by chemical derivitization of the final peptide, or by formation of the ester before coupling of either the amino acid or longer peptide chain by methods well known in the art. By way of illustration, the parent acid may be activated through the use of the condensation conditions described herein, followed by the addition of the parent alcohol, i.e., methanol, ethanol, acetate salt, if the corresponding methyl, ethyl, or acetate ester is desired, respectively. Alternatively, assuming the ester of the C-terminus acid is desired, a transesterification may be carried out at high pH as described, for example, in U.S. Pat. No. 5,663,146, the disclosure of which is hereby incorporated by reference herein in its entirety.

As will be appreciated by the skilled artisan, the peptides and peptide esters may be present in various physical forms such as pharmaceutically acceptable salts, polymorphs, and the like. Pharmaceutically acceptable addition salts includes those salts that retain the biological effectiveness and properties of the neutral compounds and that are not biologically or otherwise undesirable. The salts of the present invention can be synthesized from the parent compound, which contains a basic or acidic moiety, by conventional chemical methods. Generally, such salts can be prepared by reacting the free acid or base forms of these compounds with a stoichiometric or excess amount of the appropriate base or acid in water, or in an organic solvent, or in a mixture of the two. Generally, non-aqueous media like ether, ethyl acetate, ethanol, isopropanol, or acetonitrile are preferred. Suitable salts are known to those skilled in the art, and lists of suitable salts are found in Remington's Pharmaceutical Sciences, 17^{th} ed., Mack Publishing Company, Easton, Pa., 1985, p. 1418.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic acid salts include those derived from inorganic acids, including, but not limited to hydrochloric acid, hydrobromic acid, sulfuric acid, sulfamic acid, nitric acid, phosphoric acid and the like, and organic acids such as acetic acid, propionic acid, glycolic acid, pyruvic acid, lactic acid, oxalic acid, maleic acid, malic acid, maloneic acid, succinic acid, fumaric acid, tartaric acid, citric acid, stearic acid, ascorbic acid, benzoic acid, cinnamic acid, mandelic acid, methanesulfonic acid, ethanesulfonic acid, isethionic acid, p-toluenesulfonic acid, salicyclic acid, naturally occurring amino acids and the like.

Pharmaceutically acceptable base addition salts include those derived from inorganic bases such as sodium, potassium, lithium, ammonium, calcium, magnesium, iron, zinc, copper, manganese, aluminum salts and the like. Particularly preferred are the ammonium, potassium, sodium, calcium and magnesium salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucasamine, methylglucamine, theobromine, purines, piperizine, piperidine, N-ethylpiperidine, polyamine resins and the like. Particularly preferred organic non-toxic bases are isopropylamine, diethylamine, ethanolamine, trimethamine, dicyclohexylamine, choline, and caffeine.

It will be appreciated that the compounds of the present invention may contain one or more asymmetric carbon atoms, and may be isolated in optically active or racemic forms. Thus, all chiral, diastereomeric, racemic forms, and all geometric isomeric forms of a structure are intended, unless the specific stereochemistry or isomeric form is specifically indicated. It is well known in the art how to prepare and isolate such optically active forms. For example, mixtures of stereoisomers may be separated by standard techniques including, but not limited to, resolution of racemic forms; normal, reverse-phase, and chiral chromatography; preferential salt formation; recrystallization, solvent replacement, and the like, or by chiral synthesis either from chiral starting materials or by deliberate synthesis of target chiral centers.

The growth hormone releasing peptides described herein may be further formulated with, or attached to, a polymer. As will be appreciated by the skilled artisan, any polymer is possible, but preferably, the polymer will be biologically compatible with oral administration, and if conjugated, have a feasible attachment point for the peptide or peptide ester. The bond that conjugates or attaches the peptide to the polymer will preferably be biodegradable, such that the active ingredient is released some time following administration. In certain preferred embodiments, the peptide is conjugated to the polymer via an ester bond.

The polymers described herein, whether a discrete part of the delivery system or conjugated to a peptide, may range from alkyl to polyglycol to polysaccharide in nature and have a branched or linear structure. For example, polymers derived from alkenes that result in alkyl polymers that are branched or unbranched. In certain embodiments, the polymer is polyethylene, branched or unbranched. In more preferred embodiments, the polymer is branched polyethylene, for example, a polymer having the repeat unit —(CH₂CH(CH₃))ₙ—.

In certain embodiments, the polymer is a water-soluble polymer. Examples of water soluble polymers having applications herein include, but are not limited to, polyethylene glycol, carboxymethylcellulose, dextran, polyvinyl alcohol, polyvinyl pyrrolidone, poly-1,3-dioxolane, poly-1,3,6-trioxane, polyaminoacids (homopolymers), polypropylene glycol, copolymers of ethylene glycol/propylene glycol, ethylene/maleic anhydride copolymer, polyaminoacids, copolymer of polyethylene glycol and an amino acid, polypropylene oxide/ethylene oxide copolymers, or polyethylene glycol/thiomalic acid copolymers, hydroxypropylcellulose, hydroxypropylmethylcellulose, and hydroxyethylcellullose, hydroxypropylcellulose, hydroxypropylmethylcellulose, hydroxypropylethylcellulose, PPO/PEO block co-polymers and the like.

A polymer used in connection with the present invention can have any molecular weight. By way of general guidance, a polymer may have a molecular weight range of about 1,000 to about 1,000,000 Daltons, preferably a molecular weight range of about 20,000 to 200,000 Daltons. More preferably, the polymer has a molecular weight of about 20,000 to 100,000 Daltons.

Attachment of the peptide to the polymer may be accomplished by a variety of methods. As noted above, polymers will preferably have a functionality for ready coupling to the peptide. For example, a carboxylic acid group may be activated for reaction with the amine of a particular peptide in the presence of a base. More preferably, an alcohol or amine functionality on the polymer is used as a nucleophile to react with an activated carboxylic acid of the peptide, in the presence of base. Under such circumstances, it may be possible to append more than one peptide to a single polymer strand, if desired.

By way of further illustration, the carboxy terminus of a peptide may be reacted with an excess of ethane monomer (i.e., CH2═CHCH₃) under conditions known in the art to initiate polymerization of the monomer, providing a polyethylene polymer with the peptide attached to one end. As will be readily appreciated, the opposite end of the resultant polymer may be unsaturated, or be chemically modified to be so, in which case, a second peptide may be introduced under similar or other suitable conditions to provide a molecule of the formula:
peptide-(CH₂—CH(CH₃))—(CH₂—CH(CH₃))ₙ-peptide.

The growth hormone peptide compounds, including the parent acids and esters, or pharmaceutically acceptable salt forms thereof, optionally coupled to a polymer, may be formulated with additional pharmaceutical excipients to form an advantageous delivery system.

In certain embodiments, the delivery system will include a lipid. Lipids may be, for example, a fat, oil, wax, sterol, glycerol ether, triglyceride, saturated or unsaturated phospholipids such as phosphatidylcholine, phosphatidylglycerol, phosphatidylserine, dipalmitoylphosphatidylglycerol, dipalmitoylphosphatidylcholine, phosphatidylethanolamine, alpha-tocopherol polyethylene glycol succinate, amphilic surface active agents, a combination thereof, or any other known in the art.

The lipid are well-known in the art and may be in the form of a liposome, which is generally derived from phospholipids or other lipid substances. Liposomes are formed by mono- or multi-lamellar hydrated liquid crystals that are dispersed in an aqueous medium. However, any non-toxic, physiologically acceptable and metabolizable lipid capable of forming liposomes can be used.

Methods to form liposomes are also known in the art. The inclusion of a lipid may cause a number of effects, including changing particle characteristics, such as particle size. Another way in which a lipid can change particle characteristics is by causing formation of a microemulsion rather than micelles. Lipid-induced changes in particle characteristics can also affect bioavailability. Depending upon the composition, however, dispersions, micelles, emulsions, and the like, may result. For example, such mixtures may result upon contacting an amphiphilic substance like a phospholipid with water.

The delivery system may also contain an optional surfactant. Representative examples of long chain or high molecular weight surfactants include gelatin, casein, lecithin (phosphatides), gum acacia, cholesterol, tragacanth, polyoxyethylene alkyl ethers, e.g., macrogol ethers such as cetomacrogol 1000, polyoxyethylene castor oil derivatives, polyoxyethylene sorbitan fatty acid esters, e.g., the commercially available Tweens, polyethylene glycols, polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethylcellulose phthalate, microcrystalline cellulose, magnesium aluminum silicate, triethanolamine, polyvinyl alcohol, and polyvinylpyrrolidene (PVP). The low molecular weight include stearic acid, benzalkonium chloride, calcium stearate, glycerol monostearate, glycerol behenate, caprylates, cetostearyl alcohol, cetomacrogol emulsifying wax, and sorbitan esters. Most of these surface modifiers are known pharmaceutical excipients and are described in detail in The Handbook of Pharmaceutical Excipients, published jointly by the American Pharmaceutical Association and The Pharmaceutical Society of Great Britain, the Pharmaceutical Press, 1986, the disclosure of which is hereby incorporated herein by reference in its entirety.

The delivery system may also contain an optional organic solvent. Although any organic solvents suitable for oral administration are possible, organic alcohols such as ethanol. The delivery system also may contain water.

The delivery system may also contain additional pharmaceutical excipients including without limitation, sweeteners, flavorants, stabilizers, viscosity-increasing agent, preservatives, emulsifiers, anti-oxidants, solubilizers, fats and gums. Such excipients also include those found in Remington's Pharmaceutical Sciences, Mack Publishing Co. Easton, Pa. (1970), the disclosure of which is hereby incorporated herein by reference in its entirety. Accordingly, the term “pharmaceutically acceptable excipient” includes those excipients set forth above that are within the exercise of sound medical judgment suitable for incorporation into pharmaceutical compositions. Preferably, the delivery system described herein contains at least one excipient selected from water, lipids, liposomes, polymers, polysaccharides, organic solvents, organic acids, mineral acids, surfactants, sweeteners, flavorants, stabilizers, viscosity-increasing agent, preservatives, emulsifiers, anti-oxidants, solubilizers, fats and gums.

The amount of active compound administered will vary depending on numerous factors, e.g., the particular person treated, its age and sex, the desired therapeutic effect, the route of administration and which peptide, peptide derivatives, or combination of peptides are employed. In all instances, however, a dose effective (therapeutically effective amount) to promote release and elevation of growth hormone level in the blood of the recipient is preferably used. In general, the administration of growth hormone releasing peptides in the present delivery system may allow for lower doses of the individual growth hormone releasing compounds to be employed relative to the dose levels required for individual growth hormone releasing compounds in order to obtain a similar response, due to the synergistic effect of the combination.

By way of general guidance, however, human doses of the growth hormone releasing peptide may range from about 0.01 mg to about 1000 mg administered per day. More preferably, the dose may range from about 0.0175 mg to about 30 mg per day. Alternatively, the dosage is about 1 to about 500 mg administered two times a day. More preferably, the dosage is about 10 mg to about 300 mg, two times per day. The amount of the inactive ingredients will be known to the skilled artisan, and will depend upon the specific amount of active as well as the mode of delivery. For example, the inactive ingredients may simply be the amount necessary to fill a capsule to balance volume, or provide the appropriately sized capsule, tablet, etc. By way of general guidance, the amount (w/w) of peptide in the delivery systems described herein may range from about 0.01% to about 10%. More preferably, the amount of peptide will range from about 0.5% to about 2%. Even more preferably, the amount of peptide will range from about 0.10% to about 0.5%.

In compositions containing a lipid, it is preferably present in an amount from about 10% to greater than 50%. More preferably, the lipid is present in an amount of about 15% to about 30%. The delivery system may also contain an optional organic solvent. Although any organic solvents suitable for oral administration are possible, ethyl alcohol, glycerin, propylene glycol, and polyethylene glycol are preferred. The organic solvent is preferably present in an amount of about 1% to about 20%. The delivery system also may contain water. The amount of water will, of course, depend upon the desired composition, however, by way of general guidance, certain embodiments may contain about 30% to about 80% water.

In certain preferred embodiments, the delivery system contains water and one or more excipients selected from potassium sorbate, sodium benzoate, soy lecithin, safflower oil, PEG-40 stearate, ethyl alcohol, polysorbate 80, citric acid, chitosan, and phosphoric acid. More preferably, the delivery system contains two or more pharmaceutically acceptable excipients. Even more preferably, the delivery system contains three or more pharmaceutically acceptable excipients. By way of general guidance, the amount (w/w) of water is about 40% to about 70%; the amount of potassium sorbate is less than about 1%; the amount sodium benzoate is less than about 1%; the amount of soy lecithin is about 10% to about 40%; the amount of safflower oil is about 10% to about 20%; the amount of PEG-40 stearate is less than about 1%; the amount of ethyl alcohol is about 1% to about 3%; the amount of polysorbate 80 is about 1% to about 5%; the amount of citric acid is less than about 1%; the amount of chitosan is about 0.1% to about 3%. By way of general guidance, a preferred delivery system is set forth below.

| INGREDIENT | Per 5 kg | % w/w | 1000 g | 5000 g | units |
|---|---|---|---|---|---|
| Carbon Filtered Water | 2927.0 | 58.5400 | 585.40 | 2927 | g |
| Potassium Sorbate | 1.250 | 0.0250 | 0.25 | 1.25 | g |
| Sodium Benzoate | 0.250 | 0.0050 | 0.05 | 0.25 | g |
| Soy Lecithin | 1000.00 | 20.0000 | 200.00 | 1000 | g |
| Safflower Oil | 753.10 | 15.0620 | 150.62 | 753.1 | g |
| PEG-40 Stearate | 0.50 | 0.0900 | 0.90 | .50 | g |
| Ethyl Alcohol | 150.00 | 3.0000 | 30.00 | 150 | g |
| Polysorbate 80 | 60.00 | 2.0000 | 20.00 | 60 | g |
| GHRP-2 (free acid- | 1.00 | 0.2400 | 2.40 | 1.0 | g |
| acetate salt) | | | | | |
| Citric acid | 2.00 | 0.0400 | 0.40 | 2.0 | g |
| Chitosan | 105.00 | 1.0000 | 10.00 | 105.0 | g |
| Phosphoric Acid to pH | QS | QS | QS | QS | g |
| 4.5-5.0 | | | | | |

The peptides and delivery systems of this invention are preferably administered orally, but may be formulated in dosage forms appropriate for other routes of administration. There is, of course, wide latitude in formulation of compositions of the present invention. Solid dose forms for oral administration include, but are not limited to, capsules, tablets, pills, powders and granules. In such solid dose forms, the active compound may be mixed with inert carriers. Preferably, all components in composition are food grade materials or GRAS (Generally Recognized As Safe) materials. Information on GRAS materials can be found in Inactive Ingredient Guide, published by the U.S. Food and Drug Administration (Division of Drug Information Resources, Rockville, Md.), the disclosure of which is hereby incorporated herein by reference in its entirety. Inactive Ingredient Guide provides a listing of all inactive ingredients present in approved or conditionally approved drug products currently marketed for human use. Dosage forms also can include, as is standard practice, additional substances such as inert diluents, lubricating agents, and the like. In the case of capsules, tablets, and pills, the dose forms may also comprise buffering agents. Tablets and pills can additionally be prepared with enteric coatings.

Whether a formulation according to the invention is a liquid, semi-solid, or solid at room temperature, may depend upon the selection of components, or other concerns such as commercial viability, administration and the like. For example, a semi-solid or solid formulation is convenient for manufacturing unit doses in the form of a capsule, including both hard gelatin and soft gelatin capsules, and tablets. When the liquid or solid formulation contacts an aqueous medium, e.g., gastrointestinal liquids, the formulation becomes biologically available. The ultimate delivery mode will depend, in part, upon the solubility of the peptide ester, alternatively conjugated to a polymer, in lipids and the other excipients employed.

Compositions whose inert components are liquid at room temperature can be prepared by simply mixing the components without heating. The desired amount of the growth hormone releasing ester can be weighed out and dissolved in the mixture of inert components, with or without heating. Moderate heating, preferably less than 60° C. can be applied to hasten complete mixing of the inert components, to hasten dissolution of the peptide ester, or both. Preparation of compositions containing one or more components that are solid at room temperature is carried out at a moderately elevated temperature, preferably less than 60° C. While moderate heating can be useful, excessive heating can cause decomposition of one or more components of the formulation, as will be readily understood by one of ordinary skill in the art.

By way of further guidance, the solvents may be mixed together with a phospholipids and a surfactant, either at room temperature or heated to about 40° C. The desired peptide ester is added to this mixture and homogenized. Water and other ingredients may then be added and homogenized.

By way of further guidance, the following procedure may be followed.

| STEP | PROCEDURE |
|---|---|
| 1 | Tare a 6-Liter capacity pot |
| 2 | Load onto the pot: |
| | Carbon-Filtered Water, 2.93 Kg |
| | Polysorbate 80, 30 g. |
| | Potassium Sorbate, 1.25 g |
| | Sodium Benzoate, 0.25 g |
| | Soy Lecithin, 1.0 Kg |
| | Initiate rapid mixing with the mixer; |
| | Mix until the mixture is uniform. |
| 3 | Heat the materials in the kettle to 63-66° C. |
| | Maintain temperature. |
| 4 | Add to a 1-Liter beaker: |
| | Safflower Oil, 753.1 g |
| | PEG-40 Stearate, .50 g |
| | Heat the materials in the pot over a hotplate to 63-66° C. |
| | Mix at rapid speed with the bench top mixer. |
| 5 | Add to a 250 mL beaker: |
| | Ethyl Alcohol, 150 g |
| | Polysorbate 80, 30 g |
| | GHRP-2 (acetate salt of acid), 1.0 g |
| | Mix at rapid speed with the bench top mixer until the |
| | GHRP-2 dissolves. |
| 6 | Continue mixing the material in the 300-Gallon kettle |
| | and add the material in the 60-Liter pot to the |
| | kettle. |
| | Mix at rapid speed with the Chemineer mixer and re- |
| | circulate with the Waukesha pump for 3-5 minutes. |
| 7 | While mixing and recirculating cool the batch to 35° C. |
| | Then add to the batch: |
| | Chitosan, 105 g |
| | Citric Acid, 2.0 g |
| | Mix at rapid speed with the Chemineer mixer and re- |
| | circulate with the Waukesha pump for 3-5 minutes. |
| 8. | Take a sample from top with a sample thief. |
| | The target pH is 4.5-5.0. If the pH is above pH |
| | 5.0, then add: Phosphoric Acid in small quantities. |
| | Mix after each addition and re-measure the pH. |
| | The target pH is 4.5-5.0. If the pH is above pH 5.0, |
| | then continue adding Phosphoric acid. The target pH |
| | is 4.5-5.0. |

The methods of the present invention preferably further include contacting the composition with an aqueous medium. The composition is preferably contacted with the aqueous medium in vivo.

Accordingly, the present invention features a method of treating disease or disorder in a mammal, e.g., a human. The method includes formulating a composition containing a growth hormone peptide ester, optionally conjugated with a polymer, and a lipid, to form a system; and administering a therapeutically effective amount of the composition to the mammal. Preferably, a therapeutically effective amount of the composition is administered directly to the mammal, where it contacts an aqueous medium in vivo.

The compounds and systems described herein can advantageously be administered orally to mammals, including man, to stimulate growth hormone release. It is further suggested that continuous administration of the compounds and systems described herein will result in a sustained modulation of growth response. Thus, the compounds and systems can be used as improved functional regulators of pituitary GH secretion, thereby affecting GH or IGF-1 action.

It is known to those skilled in the art that there are many uses for growth hormone and the IGFs. Therefore administration of the compounds and systems of this invention for purposes of stimulating the release of endogenous growth hormone or IGF-1 can have, for example, the same effects or uses as growth hormone or the somatomedins themselves. The actions of the GH and IGF-1 are summarized in Goodman and Gilmans, The Pharmacological Basis of Therapeutics, 8th Ed., McGraw Hill Inc., p. 1353 (1993), the disclosure of which is hereby incorporated herein in its entirety.

By way of general guidance, the uses of growth hormone and IGF-1 include, but are not limited to, stimulating growth hormone release in elderly humans; prevention of catabolic side effects of glucocorticoids, treatment of osteoporosis, stimulation of the immune system, treatment of retardation, acceleration of wound healing, accelerating bone fracture repair, treatment of growth retardation, treating renal failure or insufficiency resulting in growth retardation, treatment of physiological short stature, including growth hormone deficient children, treating short stature associated with chronic illness, treatment of obesity and growth retardation associated with obesity, treating growth retardation associated with Prader-Willi syndrome and Turner's syndrome; accelerating the recovery and reducing hospitalization of burn patients; treatment of intrauterine growth retardation, skeletal dysplasia, hypercortisolism and Cushings syndrome; Induction of pulsatile growth hormone release; replacement of growth hormone in stressed patients; treatment of osteochondrodysplasias, Noonans syndrome, schizophrenia, depression, Alzheimer's disease, diseases of demeylination, multiple sclerosis, delayed wound healing, and psychosocial deprivation; treatment of pulmonary dysfunction and ventilator dependency; attenuation of protein catabolic response after a major operation; reducing cachexia and protein loss due to chronic illness such as cancer or AIDS; treatment of hyperinsulinemia including Type II diabetes; adjuvant treatment for ovulation induction; stimulating thymic development and prevent the age-related decline of thymic function; treatment of immuno-suppressed patients; treatment of bone marrow transplanted patients, improvement in muscle strength, mobility, diseases of muscle function, muscular dystrophy's, maintenance of skin thickness, metabolic homeostasis, enhancing renal function and hemeostasis including acute and chronic renal failure, stimulation of osteoblasts, bone remodeling, and cartilage growth; stimulation of the immune system in companion animals; growth promotion in livestock including stimulation of milk production in ruminates and wool or hair growth.

Preferably, the compounds and system described herein are used to improve muscle strength, mobility, stimulating growth hormone release in elderly humans, prevention of catabolic side effects of glucocorticoids, treatment of osteoporosis, stimulation of the immune system, and acceleration of wound healing.

Optionally, the described delivery systems can further include a growth promoting agent. Growth promoting agents include but are not limited to: TRH, diethylstilbestrol, theophylline, enkephalins, E series prostaglandins, peptides of the VIP-secretin-glucagon-GRF family and other growth hormone secretagogues such as GHRP-6; GHRP-1 as described in U.S. Pat. No. 4,411,890; and growth hormone releasing hormone (GHRH) and its analogs or growth hormone (GH) and its analogs or somatomedins including IGF-1 and IGF-2 and their analogs.

The formulations described herein can also be administered to humans in vivo as a diagnostic tool to determine whether the pituitary is capable of releasing growth hormone. Thus, the present invention may be useful in diagnostic kits.

The present invention has been shown to induce supraphysiological levels of growth hormone that may be more representative of the pulsatile GH release that takes place during adolescent growth spurts than that expected with a oral agent. For example, baseline levels of growth hormone were taken in various subjects including both men and women who were administered formulations of the present invention. Blood was then drawn to determine baseline growth hormone levels. The subjects then orally ingested a GHRP-2 peptide conjugated to a polymer matrix encased in a lipid delivery system and blood was drawn at 40 minutes and again in one hour twenty minute intervals. Test results showed increases of growth hormone levels up to roughly 90 times above baseline. One subject increased from a baseline of growth hormone level of 64 ng/dl (nanograms per deciliter) to 2560 ng/dl and in 70 minutes and further to 4120 ng/dl in 95 minutes. The reference range for adult males is 6 ng/dl to 500 ng/dl. In another example, modifying the GHRP-2 structure with an acetate attachment has proven in blood testing to induce up to 140 times over baseline and stay elevated at this level for over two hours.

All processes disclosed in association with the present invention are contemplated to be practiced on any scale, including milligram, gram, multigram, kilogram, multikilogram or commercial industrial scale. As those skilled in the art will appreciate, numerous modifications and variations of the present invention are possible in light of the above teachings. It is therefore understood that within the scope of the appended claims, the invention may be practiced otherwise than as specifically described herein, and the scope of the invention is intended to encompass all such variations.

1. A system for delivering a growth hormone releasing peptide, wherein the delivery system comprises: (a) a growth hormone releasing peptide having the structure D-Ala-D^{β}Nal-Ala-Trp-D-Phe-Lys, or a pharmaceutically acceptable salt thereof, and (b) at least one pharmaceutically acceptable excipient.

2. The delivery system according to claim 1, wherein said growth hormone releasing peptide is a salt of D-Ala-D^{β}Nal-Ala-Trp-D-Phe-Lys.

3. The delivery system according to claim 2, wherein said salt is an acid addition salt selected from the group consisting of hydrochloric acid, hydrobromic acid, sulfuric acid, sulfamic acid, nitric acid, phosphoric acid, acetic acid, propionic acid, glycolic acid, pyruvic acid, lactic acid, oxalic acid, maleic acid, malic acid, maloneic acid, succinic acid, fumaric acid, tartaric acid, citric acid, stearic acid, ascorbic acid, benzoic acid, cinnamic acid, mandelic acid, methanesulfonic acid, ethanesulfonic acid, isethionic acid, p-toluenesulfonic acid, salicyclic acid, and naturally occurring amino acids.

4. The delivery system according to claim 3, wherein said growth hormone releasing peptide is the acetate salt and is delivered in-vivo.

5. The delivery system according to claim 1, wherein the pharmaceutically acceptable excipients are selected from the group consisting of water, lipids, liposomes, polymers, polysaccharides, organic solvents, organic acids, mineral acids, surfactants, sweeteners, flavorants, stabilizers, viscosity-increasing agent, preservatives, emulsifiers, anti-oxidants, solubilizers, fats and gums.

6. The delivery system according to claim 5, wherein the pharmaceutically acceptable excipients are selected from the group consisting of water, potassium sorbate, sodium benzoate, soy lecithin, safflower oil, PEG-40 stearate, ethyl alcohol, polysorbate 80, citric acid, chitosan and phosphoric acid.

7. The delivery system according to claim 6, wherein the delivery system contains two or more excipients.

8. The delivery system according to claim 7 wherein the growth hormone releasing peptide is the acetate salt of D-Ala-D^{β}Nal-Ala-Trp-D-Phe-Lys.

9. A system for delivering a growth hormone releasing peptide, wherein the delivery system comprises a growth hormone releasing peptide having the structure D-Ala-D^{β}Nal-Ala-Trp-D-Phe-Lys-OR, or a pharmaceutically acceptable salt form thereof, wherein R is a group that provides a free hydroxyl after being administered to a subject; and at least one pharmaceutically acceptable excipient.

10. The delivery system according to claim 9, wherein said growth releasing peptide is a salt of D-Ala-D^{β}Nal-Ala-Trp-D-Phe-Lys-OR.

11. The delivery system according to claim 9, wherein R is selected from the group consisting of C₁-C₁₀ alkyl, aryl, alkylaryl and acetate.

12. The delivery system according to claim 11, wherein R is selected from the group consisting of methyl, ethyl, propyl, isopropyl, butyl, iso-butyl, sec-butyl, tert-butyl, cyclopropyl, cyclobutyl, cyclopentyl, cyclohexyl, phenyl, benzyl, and phenethyl.

13. The delivery system according to claim 12, wherein R is methyl or ethyl and said growth hormone releasing peptide is delivered in-vivo.

14. The delivery system according to claim 9, wherein the pharmaceutically acceptable excipients are selected from the group consisting of water, lipids, liposomes, polymers, polysaccharides, organic solvents, organic acids, mineral acids, surfactants, sweeteners, flavorants, stabilizers, viscosity-increasing agent, preservatives, emulsifiers, anti-oxidants, solubilizers, fats and gums.

15. The delivery system according to claim 14, wherein the pharmaceutically acceptable excipients are selected from the group consisting of water, potassium sorbate, sodium benzoate, soy lecithin, safflower oil, PEG-40 stearate, ethyl alcohol, polysorbate 80, citric acid, chitosan and phosphoric acid.

16. The delivery system according to claim 15, wherein the delivery system comprises two or more pharmaceutically acceptable excipients.

17. A method for forming a system for delivering a growth hormone releasing peptide which comprises providing a growth releasing peptide or a pharmaceutically acceptable salt form thereof and dispersing the growth hormone releasing peptide in at least one pharmaceutically acceptable excipients under suitable conditions to form a delivery system.

18. The method according to claim 17, wherein the peptide is selected from the group consisting of a salt of Ala-D^{β}Nal-Ala-Trp-D-Phe-Lys and Ala-D^{β}Nal-Ala-Trp-D-Phe-Lys-OR, wherein R is C₁-C₁₀ alkyl; and the pharmaceutically acceptable excipients are selected from the group consisting of water, lipids, liposomes, polymers, polysaccharides, organic solvents, organic acids, mineral acids, surfactants, sweeteners, flavorants, stabilizers, viscosity-increasing agent, preservatives, emulsifiers, anti-oxidants, solubilizers, fats and gums.

19. A method of releasing and elevating growth hormone levels, which comprises administering a therapeutically effective amount of the delivery system according to claim 1 to a subject.

20. The method according to claim 19, wherein the releasing of growth hormone improves muscle strength, improves mobility, stimulates growth hormone release in elderly humans, prevents catabolic side effects of glucocorticoids, prevents osteoporosis, stimulates the immune system, and accelerates wound healing.
